(19) United States
(12) Patent Application Publication
(10) Pub. No.: US 2006/0211754 A1
(43) Pub. Date: Sep. 21, 2006

(54) Compositions comprising N-propanoyl derivatives of amino acids, aminocarbohydrates and derivatives thereof

(76) Inventors: Ruey J. Yu, Chalfont, PA (US); Eugene J. Van Scott, Abington, PA (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 11/375,570

(22) Filed: Mar. 15, 2006

Related U.S. Application Data
(60) Provisional application No. 60/661,921, filed on Mar. 16, 2005.

(51) Int. Cl. A61K 31/4172; A61K 31/198

(52) U.S. Cl. 514/400; 514/562; 514/563

(57) ABSTRACT

The embodiments relate to compositions comprising therapeutically effective amounts of at least one N-propanoyl derivative of amino acids, aminocarbohydrates, and derivatives thereof. The compositions are useful the prevention and treatment of symptoms or syndromes associated with nervous, vascular, musculoskeletal, or cutaneous systems. The compositions may be topically or systemically administered to a subject in need thereof.

This application claims priority under 35 U.S.C. §119 to Provisional Patent Application No. 60/661,921, filed on Mar. 16, 2005, the disclosure of which is herein incorporated by reference in its entirety.

### FIELD OF THE INVENTION

The embodiments relate to compositions comprising N-propanoyl derivatives of amino acids, aminocarbohydrates or derivatives, and to the systemic or topical administration of the above compositions to a subject for therapeutic or preventive treatment to alleviate or improve disorders, symptoms, or syndromes associated with the (A) nervous, (B) vascular, (C) musculoskeletal or (D) cutaneous systems.

### BACKGROUND

Conventional compositions and methods for the treatment of disorders, symptoms, or syndromes associated with the nervous, vascular, musculoskeletal or cutaneous systems present certain disadvantages. Conventional treatment options include, for example, the use of aspirin, N-Acetyl derivatives of amino acids and aminocarbohydrates, and Corticosteroids. Aspirin is commonly used for temporary relief of pain and inflammation of arthritis and bursitis. The most common adverse reactions are stomach irritation and gastrointestinal bleeding. N-Acetyl derivatives of amino acids and aminocarbohydrates have been used for topical treatment of various cosmetic conditions and dermatological disorders. The therapeutic effects, however, sometimes are limited due to lower lipid solubility, poor penetration and lower bioavailability to target tissues. Corticosteroids such as prednisone and non-steroidal antiinflammatory drugs such as ibuprofen, naproxen, tolmetin and sulindac also may be used for temporary relief of arthritis. These drugs, however, also may cause adverse side effects on long-term use.

Additional compositions alleged to be suitable for treatment of disorders, symptoms, or syndromes associated with the nervous, vascular, musculoskeletal or cutaneous systems are provided by the following references:

PCT Application No. PCT/US96/16534, filed Oct. 16, 1996, entitled “Topical Compositions Containing N-Acetyl-cysteine and Odor Masking Materials,” describes topical compositions comprising from 0.01% to 50% of N-acetyl-cysteine or a derivative of N-acetyl-cysteine, from 0.01% to 0.5% of an odor masking material, and a topical carrier to improve the appearance of skin.

EP patent No. 0308278 describes the use of N-acetyl-proline, N-acetyl-hydroxyproline, N-propanoyl-proline and N-propanoyl-hydroxyproline as free acid or salt form in cosmetic products for stretch marks, skin elasticity, anti-wrinkles and as emollients.

U.S. Pat. No. 6,159,485 entitled “N-Acetyl Aldosamines, N-Acetylamino Acids and Related N-Acetyl Compounds and Their Topical Use”, U.S. Pat. No. 6,524,593 B1 entitled “N-Acetyl Aldosamines and Related N-Acetyl Compounds, and Their Topical Use,” and U.S. Pat. No. 6,808,716 B2 entitled “N-Acetylamino Acids, Related N-Acetyl Compounds and Their Topical Use,” describe and claim the use of compositions comprising N-acetylamino acids and N-acetyl aldosamines for topical treatment of cosmetic conditions and dermatological disorders.

U.S. Pat. No. 6,824,786 B2 entitled “Compositions Comprising Phenyl-Glycine Derivatives” describes and claims the compositions and use of the compositions comprising phenyl-glycine derivatives for treating cosmetic conditions and dermatological disorders. The disclosures of each of the aforementioned United States patents are incorporated by reference herein in their entireties.

Suitable and effective treatment options do not exist for some disorders, symptoms, or syndromes, especially those associated with the nervous system. For example, there are no presently available pharmaceutical drugs that can reverse or even stop the progression of the Alzheimer's disease. A subject or patient having Alzheimer's disease usually takes various vitamins and medications to slow down the progression of short term memory loss associated with the disease. Typically administered vitamins and drugs, such as donepezil, memantine, melatonin, lipoic acid, selenium, and folic acid, have minimal benefits for slowing the progression of mental deterioration, and provide little improvement in quality of life. The patient's memory usually may be deteriorated to a point where recognition of family members and care givers is minimal. The patient may also sleep for long hours, such as more than 12 hours daily. Furthermore, episodes of urinary and bowel incontinence may occur daily.

The description herein of disadvantages and problems associated with known compositions, and methods is in no way intended to limit the scope of the embodiments described in this document to their exclusion. Indeed, certain embodiments may include one or more known composition, compound, or method without suffering from the so-noted disadvantages or problems.

### SUMMARY

It is thus a feature of the embodiments to provide compositions and methods for the therapeutic or preventive treatment to alleviate or improve disorders, symptoms, or syndromes associated with the nervous, vascular, musculoskeletal or cutaneous systems. In accordance with this feature and other features readily apparent to those skilled in the art, the embodiments provide compositions comprising therapeutically effective amounts of N-propanoyl derivative of amino acids, aminocarbohydrates, derivatives thereof, and mixtures thereof.

The embodiments also provide methods of using compositions comprising therapeutically effective amounts of N-propanoyl derivative of amino acids, aminocarbohydrates, derivatives thereof, and mixtures thereof for the prevention and treatment of symptoms or syndromes associated with nervous, vascular, musculoskeletal, or cutaneous systems. The compositions may be topically or systemically administered to a subject in need thereof.

These and other features of various embodiments will become readily apparent to those skilled in the art upon review of the detailed description that follows.

### DETAILED DESCRIPTION

For the purposes of promoting an understanding of the embodiments described herein, reference will be made to preferred embodiments and specific language will be used to describe the same. The terminology used herein is for the purpose of describing particular embodiments only, and is not intended to limit the scope of the present invention. As used throughout this disclosure, the singular forms “a,” “an,” and “the” include plural reference unless the context clearly dictates otherwise. Thus, for example, a reference to “a composition” includes a plurality of such compositions, as well as a single composition, and a reference to “a therapeutic agent” is a reference to one or more therapeutic and/or pharmaceutical agents and equivalents thereof known to those skilled in the art, and so forth.

The present inventors have discovered that N-propanoyl derivatives of amino acids or aminocarbohydrates, and derivatives thereof are therapeutically effective or beneficial, by systemic or topical administration, for prevention or treatment to alleviate or improve symptoms or syndromes associated with the nervous, vascular, musculoskeletal or cutaneous systems. N-propanoyl derivatives of amino acids and aminocarbohydrates are more lipid-soluble, more bioavailable to target tissues, and more therapeutically effective than N-acetyl derivatives of amino acids and aminocarbohydrates.

Representative compounds useful in particularly preferred embodiments include, N-propanoyl-glucosamine, N-propanoyl-galactosamine, N-propanoyl-arginine, N-propanoyl-glutamic acid, N-propanoyl-glutamic acid diethyl ester, N-propanoyl-glutamine, N-propanoyl-prolinamide, N-propanoyl-proline ethyl ester and N-propanoyl-creatinine. N-propanoyl-proline, N-propanoyl-hydroxyproline, and compositions containing these compounds preferably are expressly excluded from the scope of the embodiments.

The N-propanoyl derivatives of the embodiments may be divided into two groups: (A) N-propanoylamino acids; and (B) N-propanoyl aminocarbohydrates.

(A). N-Propanoylamino Acids

N-Propanoylamino acids preferably are N-substituted derivatives of amino acids, and may be represented by the following generic structure:
R₁CH(NHR₂)(CH₂)ₙCOR₃
where R₁ is H, an alkyl, aralkyl or aryl group having 1 to 14 carbon atoms, and R₁ may also carry OH, SH, SCH₃, NH₂, NHR₂, CONH₂, NHCONH₂, NHC(═NH)NH₂, NHC(═NR₂)NH₂, imidazole, pyrrolidine or other heterocyclic group; n is an integer from 0 to 5; R₂ is independently an propanoyl group having COCH₂CH₃; R₃ is NH₂, OR₄; R₄ is H, an alkyl, aralkyl or aryl group having 1 to 9 carbon atoms; and the H attached to any carbon atom may be substituted by I, F, Cl, Br, OH or alkoxy group having 1 to 9 carbons. The N-propanoylamino acids may be present as a free acid, salt, amide, ester or lactone form, as stereoisomers such as D, L, or DL form, or non-stereoisomers such as N-propanoyl-glycine.

Among commonly known N-propanoylamino acids, N-propanoyl-proline cannot be represented by the above generic structure because the alpha amino group is part of the heterocyclic pyrrolidine ring. Therefore, N-propanoyl derivatives of proline such as N-propanoyl-proline, N-propanoyl-prolinamide and N-propanoyl-proline esters will be represented by their chemical names. In the same manner, chemical names will be used if the compounds of the embodiments cannot be covered by the above generic structure.

Most amino acids have only one amino group attached to the alpha carbon, and have only one propanoyl group attached to the amino group such as N-propanoyl-glycine. Some amino acids, such as lysine, omithine, arginine, histidine and tryptophan have additional amino, imino, or guanidino group in addition to the alpha amino group, and can form N, N′-dipropanoylamino acids, such as N, N′-dipropanoyl-lysine. The preferred N-propanoyl derivative is mono-N-propanoylamino acid, such as alpha N-propanoyl-lysine.

Representative N-propanoylamino acids are N-Propanoyl derivatives of common amino acids and N-Propanoyl derivatives of related amino acids. Representative N-Propanoyl derivatives of common amino acids include, but are not limited to, N-propanoyl-alanine, N-propanoyl-arginine, N,N′-dipropanoyl-arginine, N-propanoyl-asparagine, N-propanoyl-aspartic acid, N-propanoyl-cysteine, N-propanoyl-glycine, N-propanoyl-glutamic acid, N-propanoyl-glutamine, N-propanoyl- histidine; N,N -dipropanoyl-histidine, N-propanoyl-isoleucine, N-propanoyl-leucine, N-propanoyl-lysine; N,N-dipropanoyl-lysine, N-propanoyl-methionine, N-propanoyl-phenylalanine, N-propanoyl-proline, N-propanoyl-serine, N-propanoyl-threonine, N-propanoyl-tryptophan, N,N-dipropanoyl-tryptophan, N-propanoyl-tyrosine, and N-propanoyl-valine.

The following are related compounds that may or may not be represented by the above generic structure: N-propanoyl-β-alanine, N-propanoyl-γ-aminobutanoic acid, N-propanoyl-β-aminoisobutanoic acid, N-propanoyl-citrulline, N-propanoyl-dopa (N-propanoyl-3,4-dihydroxyphenylalanine), N-propanoyl-homocysteine, N-propanoyl-homoserine, N-propanoyl-ornithine, and N,N′-dipropanoyl-ornithine.

The N-propanoylamino acids and related compounds disclosed herein may be present as a free acid, salt, or partial salt with organic or inorganic alkali, lactone, amide, or ester. Further, the N-propanoylamino acids and related compounds disclosed herein may be present in a stereoisomeric or non-stereoisomeric form. As an illustration, N-propanoyl-proline includes, for example, N-propanoyl-L-proline; N-propanoyl-L-proline sodium salt; N-propanoyl-L-prolinamide, N-propanoyl-L-proline methyl ester, N-propanoyl-L-proline ethyl ester, N-propanoyl-L-proline propyl ester and N-propanoyl-L-proline isopropyl ester.

(B) N-Propanoylaminocarbohydrates

The preferred N-propanoyl derivatives of aminocarbohydrates useful in the embodiments may be represented by the following generic structure:
R₁(CHOH)ₙ(CHNHR₂)R₃
where R₁ is selected from the group consisting of H, I, F, Cl, Br, CHO, CONH₂, COOR₄, an alkyl, alkoxyl, aralkyl or aryl group having 1 to 9 carbon atoms; n is an integer, preferably from 1-9; R₂ is a propanoyl group having COCH₂CH₃; R₃ is selected from the group consisting of H, CHO, CONH₂, and COOR₄; R₄ is independently selected from the group consisting of H, an alkyl, aralkyl, or aryl group having 1 to 9 carbon atoms; the H attached to any carbon atom may be substituted by I, F, Cl, Br, SH, CHO, CONH₂, NH₂, or an alkyl, alkoxyl, aralkyl, or aryl group having 1 to 9 carbon atoms.

The N-propanoylaminocarbohydrate may be present as free acid, salt, partial salt, amide, ester, lactone, straight, branched or cyclic form, as stereoisomer such as D, L, or DL, or non-stereoisomer. A typical cyclic form of an N-propanoyl derivative of the aminocarbohydrate is a five member ring (furanose form) or six member ring (pyranose form) of the aminocarbohydrate moiety.

The aminocarbohydrate may have more than one amino group in the molecule. In a preferred embodiment, the aminocarbohydrate has only one amino group. Glucosamine, which can form N-propanoyl-glucosamine, is an example of an aminocarbohydrate that has only one amino group.

Representative embodiments include, but are not limited to, are N-propanoyl-aminocarbohydrates including, but are not limited to, N-propanoyl-glycerosamine, N-propanoyl-erythrosamine, N-propanoyl-threosamine, N-propanoyl-ribosamine, N-propanoyl-arabinosamine, N-propanoyl-xylosamine, N-propanoyl-lyxosamine, N-propanoyl-allosamine, N-propanoyl-altrosamine, N-propanoyl-glucosamine, N-propanoyl-mannosamine, N-propanoyl-gulosamine, N-propanoyl-idosamine, N-propanoyl-galactosamine, N-propanoyl-talosamine, N-propanoyl-alloheptosamine, N-propanoyl-altroheptosamine, N-propanoyl-glucoheptosamine, N-propanoyl-mannoheptosamine, N-propanoyl-guloheptosamine, N-propanoyl-idoheptosamine, N-propanoyl-galactoheptosamine, N-propanoyl-taloheptosamine, N-propanoyl-glyceraminic acid, N-propanoyl-erythrosaminic acid, N-propanoyl-threosaminic acid, N-propanoyl-ribosaminic acid, N-propanoyl-arabinosaminic acid, N-propanoyl-xylosaminic acid, N-propanoyl-lyxosaminic acid, N-propanoyl-allosaminic acid, N-propanoyl-altrosaminic acid, N-propanoyl-glucosaminic acid, N-propanoyl-mannosaminic acid, N-propanoyl-gulosaminic acid, N-propanoyl-idosaminic acid, N-propanoyl-galactosaminic acid, N-propanoyl-talosaminic acid, N-propanoyl-alloheptosaminic acid, N-propanoyl-altroheptosaminic acid, N-propanoyl-glucoheptosaminic acid, N-propanoyl-mannoheptosaminic acid, N-propanoyl-guloheptosaminic acid, N-propanoyl-idoheptosaminic acid, N-propanoyl-galactoheptosaminic acid, N-propanoyl-taloheptosaminic acid, N-propanoyllactosamine, N-propanoylmuramic acid, N-propanoylneuramine, N-propanoylneuramin lactose, N-propanoyl-neuraminic acid, N-propanoyl-glycerosylamine, N-propanoyl-erythrosylamine, N-propanoyl-threosylamine, N-propanoyl-ribosylamine, N-propanoyl-arabinosylamine, N-propanoyl-xylosylamine, N-propanoyl-lyxosylamine, N-propanoyl-allosylamine, N-propanoyl-altrosylamine, N-propanoyl-glucosylamine, N-propanoyl-mannosylamine, N-propanoyl-gulosylamine, N-propanoyl-idosylamine, N-propanoyl-galactosylamine, N-propanoyl-talosylamine, N-propanoyl-alloheptosylamine, N-propanoyl-altroheptosylamine, N-propanoyl-glucoheptosylamine, N-propanoyl-mannoheptosylamine, N-propanoyl-guloheptosylamine, N-propanoyl-idoheptosylamine, N-propanoyl-galactoheptosylamine, N-propanoyl-taloheptosylamine, and N-propanoyl-aminocyclitols.

The N-propanoyl derivatives of amino acids and aminocarbohydrates of the preferred embodiments include N-propanoyl-prolinamide, N-propanoyl-proline ethyl ester, N-propanoyl-proline propyl ester, N-propanoyl-glutamic acid, N-propanoyl-glutamic acid diethyl ester, N-propanoyl-glutamine, N-propanoyl-glutamine ethyl ester, N-propanoyl-glucosamine, N-propanoyl-galactosamine and N-propanoyl-creatinine.

Compositions comprising N-Propanoyl derivatives of amino acids and aminocarbohydrates are therapeutically beneficial or effective for prevention or treatment to alleviate or improve symptoms or syndromes associated with the nervous, vascular, musculoskeletal or cutaneous systems. The compositions of the preferred embodiments comprise a therapeutically effective amount of N-propanoyl derivative of amino acid or aminocarbohydrate and may be in a pharmaceutically acceptable vehicle for topical and systemic treatment of disorders associated with nervous, vascular, musculoskeletal, or cutaneous systems.

Compositions comprising N-propanoyl derivatives may be formulated in any manner suited for topical or systemic administration to a subject or patient. Examples of suitable formulations that may be used for topical or systemic administration include, but are not limited to, a solution, gel, lotion, cream, ointment, shampoo, spray, stick, powder, masque, mouth rinse or wash, vaginal gel or preparation, or other form acceptable for use on skin, nail, hair, oral mucosa, vaginal or anal mucosa, mouth or gums.

Compositions of N-Propanoyl derivatives of amino acids and aminocarbohydrates preferably comprise at least one compound selected from the group consisting of N-propanoyl derivatives of amino acids, aminocarbohydrates, and derivatives thereof, which may be in the form of a free acid, ester, amide, lactone, or salt. Compositions comprising an N-propanoyl derivative of an amino acid or aminocarbohydrate may be administered topically or systemically to a human subject in need thereof.

For topical administration, a composition comprising an N-propanoyl derivative of amino acid or aminocarbohydrate may be topically applied one to three times, preferably twice daily, to the lesions or the cutaneous sites associated with disorders or diseases. The topical application may continue until the symptom or disease has been eradicated or substantially improved. The treatment period depends on the condition or severity of the disorder or disease, and also depends on the individual subject.

Those of ordinary skill in the art will recognize how to incorporate the N-propanoyl derivatives of the embodiments into formulations suitable for topical administration to a subject. As an example, to prepare a solution composition, at least one N-propanoyl derivative of the present invention may be dissolved in a solution prepared from water, ethanol, propylene glycol, butylene glycol, and/or other topically acceptable vehicle. The concentration of a single N-propanoyl derivative or the total concentration of all N-propanoyl derivatives where the composition comprises more than one N-propanoyl derivative, may range from 0.01 to 99.9% by weight of the total composition, with preferred concentration of from 0.1 to 50% by weight of the total composition and with more preferred concentration of from 1 to 20% by weight of the total composition.

A topical composition in lotion, cream, or ointment form, may be prepared, for example, by first dissolving the N-propanoyl derivative in water, ethanol, propylene glycol, and/or other vehicle. The solution thus obtained may be mixed with a desired base or pharmaceutically acceptable vehicle to make lotion, cream or ointment. Concentrations of the N-propanoyl derivative may be the same as described above.

A topical composition of the preferred embodiments also may be formulated in a gel or shampoo form. A typical gel composition may be formulated by the addition of a gelling agent, such as chitosan, methyl cellulose, ethyl cellulose, polyvinyl alcohol, polyquaterniums, hydroxyethylcellulose, hydroxypropylcellulose, hydroxypropylmethylcellulose, carbomer or ammoniated glycyrrhizinate to a solution comprising the N-propanoyl derivative. The preferred concentration of the gelling agent may range from 0.1 to 4 percent by weight of the total composition. In the preparation of shampoo, the N-propanoyl derivative may be first dissolved in water or propylene glycol, and the solution thus obtained may be mixed with a shampoo base. Concentrations of the N-propanoyl derivative used in gel or shampoo form are the same as described above.

Systemic administration includes, but is not limited to, injection, infusion, oral administration. The preferred route is oral administration. A composition comprising the N-propanoyl derivative may be taken orally one to three times, preferably twice daily, for prevention or treatment of disorders and diseases associated with nervous, vascular, musculoskeletal or cutaneous system. The oral administration may continue until the symptom or disease has been eradicated or substantially improved. The symptoms or disorders include, for example, pains, pruritus, inflammation, erythema, dermatitis, acne, eczema, dementia, Alzheimer's disease, joint pain or swelling, and arthritis.

The N-propanoyl derivatives may be formulated for oral administration or for parenteral injections for systemic use. In oral preparations the N-propanoyl derivative may be formulated in tablet form or in gelatin capsules with or without mixing with gelatin powder. Each tablet or capsule may contain from about 10 to about 500 mg of the N-propanoyl derivative of amino acids or aminocarbohydrates as free acid, salt, amide, ester or lactone form. The oral dose may range from between about 10 mg to about 1000 mg daily, and preferably from between about 100 mg to about 500 mg daily. This oral dose may be divided into portions to be taken at multiple times per day. For example, the oral dose may be divided into equal amounts of 50 mg to 250 mg when taken twice daily. For parenteral injections, the N-propanoyl derivative may be prepared under sterilized conditions in 1 to 30%, preferably 1 to 10%, concentration in water, propylene glycol and/or non-aqueous vehicle.

In another embodiment, the composition may further comprise an additional cosmetic, pharmaceutical, or other agent to achieve synergetic or synergistic effects. To prepare a topical combination composition, a cosmetic, pharmaceutical or other agent is incorporated into any one of the above compositions by dissolving or mixing the agent into the formulation. Other forms of compositions for delivery of the N-propanoyl derivative of the present invention are readily recognized by those skilled in the art.

Vitamins, cosmetics, pharmaceutical and/or other agents may be used topically or taken systemically at the same time, sequentially, or in combination to complement or enhance therapeutic effects of N-propanoyl derivatives of amino acids or aminocarbohydrates. Examples of such agents include abacavir, acebutolol, acetaminophen, acetaminosalol, acetazolamide, acetohydroxamic acid, acetylsalicylic acid, acitretin, aclovate, acrivastine, actiq, acyclovir, adapalene, adefovir dipivoxil, adenosine, albuterol, alfuzosin, allopurinol, alloxanthine, almotriptan, alprazolam, alprenolol, aluminum acetate, aluminum chloride, aluminum chlorohydroxide, aluminum hydroxide, amantadine, amiloride, aminacrine, aminobenzoic acid (PABA), aminocaproic acid, aminosalicylic acid, amiodarone, amitriptyline, amlodipine, amocarzine, amodiaquin, amorolfine, amoxapine, amphetamine, ampicillin, anagrelide, anastrozole, anthralin, apomorphine, aprepitant, arbutin, aripiprazole, ascorbic acid, ascorbyl palmitate, atazanavir, atenolol, atomoxetine, atropine, azathioprine, azelaic acid, azelastine, azithromycin, bacitracin, beclomethasone dipropionate, bemegride, benazepril, bendroflumethiazide, benzocaine, benzonatate, benzophenone, benzoyl peroxide, benztropine, bepridil, betamethasone dipropionate, betamethasone valerate, brimonidine, brompheniramine, bupivacaine, buprenorphine, bupropion, burimamide, butenafine, butoconazole, cabergoline, caffeic acid, caffeine, calcipotriene, camphor, candesartan cilexetil, capsaicin, carbamazepine, carbamide peroxide, cefditoren pivoxil, cefepime, cefpodoxime proxetil, celecoxib, cetirizine, cevimeline, chitosan, chlordiazepoxide, chlorhexidine, chloroquine, chlorothiazide, chloroxylenol, chlorpheniramine, chlorpromazine, chlorpropamide, ciclopirox, cilostazol, cimetidine, cinacalcet, ciprofloxacin, citalopram, citric acid, cladribine, clarithromycin, clemastine, clindamycin, clioquinol, clobetasol propionate, clomiphene, clonidine, clopidogrel, clotrimazole, clozapine, coal tar, coal tar extracts (LCD), cocaine, codeine, cromolyn, crotamiton, cyclizine, cyclobenzaprine, cycloserine, cytarabine, dacarbazine, dalfopristin, dapsone, daptomycin, daunorubicin, deferoxamine, dehydroepiandrosterone, delavirdine, desipramine, desloratadine, desmopressin, desoximetasone, dexamethasone, dexmedetomidine, dexmethylphenidate, dexrazoxane, dextroamphetamine, diazepam, dicyclomine, didanosine, dihydrocodeine, dihydromorphine, diltiazem, 6,8-dimercaptooctanoic acid (dihydrolipoic acid), diphenhydramine, diphenoxylate, dipyridamole, disopyramide, dobutamine, dofetilide, dolasetron, donepezil, dopa esters, dopamide, dopamine, dorzolamide, doxepin, doxorubicin, doxycycline, doxylamine, doxypin, duloxetine, dyclonine, econazole, eflornithine, eletriptan, emtricitabine, enalapril, ephedrine, epinephrine, epinine, epirubicin, eptifibatide, ergotamine, erythromycin, escitalopram, esmolol, esomeprazole, estazolam, estradiol, ethacrynic acid, ethinyl estradiol, etidocaine, etomidate, famciclovir, famotidine, felodipine, fentanyl, ferulic acid, fexofenadine, flecainide, fluconazole, flucytosine, fluocinolone acetonide, fluocinonide, 5-fluorouracil, fluoxetine, fluphenazine, flurazepam, fluvoxamine, formoterol, furosemide, galactarolactone, galactonic acid, galactonolactone, galantamine, gatifloxacin, gefitinib, gemcitabine, gemifloxacin, glucarolactone, gluconic acid, gluconolactone, glucuronic acid, glucuronolactone, glycolic acid, griseofulvin, guaifenesin, guanethidine, N-guanylhistamine, haloperidol, haloprogin, hexylresorcinol, homatropine, homosalate, hydralazine, hydrochlorothiazide, hydrocortisone, hydrocortisone 21-acetate, hydrocortisone 17-butyrate, hydrocortisone 17-valerate, hydrogen peroxide, hydromorphone, hydroquinone, hydroquinone monoether, hydroxyzine, hyoscyamine, hypoxanthine, ibuprofen, ichthammol, idarubicin, imatinib, imipramine, imiquimod, indinavir, indomethacin, irbesartan, irinotecan, isoetharine, isoproterenol, itraconazole, kanamycin, ketamine, ketanserin, ketoconazole, ketoprofen, ketotifen, kojic acid, labetalol, lactic acid, lactobionic acid, lamivudine, lamotrigine, lansoprazole, letrozole, leuprolide, levalbuterol, levofloxacin, lidocaine, linezolid, lobeline, loperamide, losartan, loxapine, lysergic diethylamide, mafenide, malic acid, maltobionic acid, mandelic acid, maprotiline, mebendazole, mecamylamine, meclizine, meclocycline, memantine, menthol, meperidine, mepivacaine, mercaptopurine, mescaline, metanephrine, metaproterenol, metaraminol, metformin, methadone, methamphetamine, methotrexate, methoxamine, methyidopa esters, methyidopamide, 3,4-methylenedioxymethamphetamine, methyllactic acid, methyl nicotinate, methylphenidate, methyl salicylate, metiamide, metolazone, metoprolol, metronidazole, mexiletine, miconazole, midazolam, midodrine, miglustat, minocycline, minoxidil, mirtazapine, mitoxantrone, moexiprilat, molindone, monobenzone, morphine, moxifloxacin, moxonidine, mupirocin, nadolol, naftifine, nalbuphine, nalmefene, naloxone, naproxen, nefazodone, nelfinavir, neomycin, nevirapine, nicardipine, nicotine, nifedipine, nimodipine, nisoldipine, nizatidine, norepinephrine, nystatin, octopamine, octreotide, octyl methoxycinnamate, octyl salicylate, ofloxacin, olanzapine, olmesartan medoxomil, olopatadine, omeprazole, ondansetron, oxiconazole, oxotremorine, oxybenzone, oxybutynin, oxycodone, oxymetazoline, padimate O, palonosetron, pantothenic acid, pantoyl lactone, paroxetine, pemoline, penciclovir, penicillamine, penicillins, pentazocine, pentobarbital, pentostatin, pentoxifylline, pergolide, perindopril, permethrin, phencyclidine, pheneizine, pheniramine, phenmetrazine, phenobarbital, phenol, phenoxybenzamine, phentolamine, phenylephrine, phenylpropanolamine, phenytoin, physostigmine, pilocarpine, pimozide, pindolol, pioglitazone, pipamazine, piperonyl butoxide, pirenzepine, podofilox, podophyllin, povidone iodine, pramipexole, pramoxine, prazosin, prednisone, prenalterol, prilocaine, procainamide, procaine, procarbazine, promazine, promethazine, promethazine propionate, propafenone, propoxyphene, propranolol, propylthiouracil, protriptyline, pseudoephedrine, pyrethrin, pyrilamine, pyrimethamine, quetiapine, quinapril, quinethazone, quinidine, quinupristin, rabeprazole, reserpine, resorcinol, retinal, 13-cis retinoic acid, retinoic acid, retinol, retinyl acetate, retinyl palmitate, ribavirin, ribonic acid, ribonolactone, rifampin, rifapentine, rifaximin, riluzole, rimantadine, risedronic acid, risperidone, ritodrine, rivastigmine, rizatriptan, ropinirole, ropivacaine, salicylamide, salicylic acid, salmeterol, scopolamine, selegiline, selenium sulfide, serotonin, sertindole, sertraline, shale tar, sibutramine, sildenafil, sotalol, streptomycin, strychnine, sulconazole, sulfabenz, sulfabenzamide, sulfabromomethazine, sulfacetamide, sulfachlorpyridazine, sulfacytine, sulfadiazine, sulfadimethoxine, sulfadoxine, sulfaguanole, sulfalene, sulfamethizole, sulfamethoxazole, sulfanilamide, sulfapyrazine, sulfapyridine, sulfasalazine, sulfasomizole, sulfathiazole, sulfisoxazole, sulfur, tadalafil, tamsulosin, tartaric acid, tazarotene, tegaserol, telithromycin, telmisartan, temozolomide, tenofovir disoproxil, terazosin, terbinafine, terbutaline, terconazole, terfenadine, tetracaine, tetracycline, tetrahydrozoline, theobromine, theophylline, thiabendazole, thioctic acid (lipoic acid), thioridazine, thiothixene, thymol, tiagabine, timolol, tinidazole, tioconazole, tirofiban, tizanidine, tobramycin, tocainide, tolazoline, tolbutamide, tolnaftate, tolterodine, tramadol, tranylcypromine, trazodone, triamcinolone acetonide, triamcinolone diacetate, triamcinolone hexacetonide, triamterene, triazolam, triclosan, triflupromazine, trimethoprim, trimipramine, tripelennamine, triprolidine, tromethamine, tropic acid, tyramine, undecylenic acid, urea, urocanic acid, ursodiol, vardenafil, venlafaxine, verapamil, vitamin E acetate, voriconazole, warfarin, wood tar, xanthine, zafirlukast, zaleplon, zinc pyrithione, ziprasidone, zolmitriptan, and zolpidem.

Topical agents that may be readily added to or used in conjunction with the above formulations of N-propanoyl derivatives of amino acids or aminocarbohydrates of the preferred embodiments include, but are not limited to: hydroxyacids, ketoacids and related compounds; phenyl alpha acyloxyalkanoic acids and derivatives; N-acetyl-aldosamines, N-acetylamino acids and related N-acetyl compounds; local analgesics and anesthetics; antiacne agents; antibacterials; antiyeast agents; antifungal agents; antiviral agents; antidandruff agents; antidermatitis agents; antihistamine agents; antipruritic agents; antiemetics; antimotion sickness agents; antiinflammatory agents; antihyperkeratotic agents; antiperspirants; antipsoriatic agents; antiseborrheic agents; hair conditioners and hair treatment agents; antiaging and antiwrinkle agents; sunblock and sunscreen agents; skin lightening agents; depigmenting agents; astringents; cleansing agents; corn, callus and wart removing agents; topical cardiovascular agents; vitamins; corticosteroids; tanning agents; hormones; retinoids; gum disease or oral care agents.

N-propanoyl derivatives of amino acids or aminocarbohydrates, and derivatives thereof, have broad utilities for prevention or treatment to alleviate or improve symptoms or syndromes associated with the nervous system. Symptoms and syndromes associated with the nervous system include, but are not limited to: (1) dementia and Alzheimer's disease: progressive loss of memory, shrinkage and atrophy of cerebral cortex, tangles of fibers in nerve cells, senile plaques of amyloid, decreased choline acetyltransferase enzyme; (2) carpal tunnel syndrome: weakness, pain, tingling, numbness, burning in palm and fingers; (3) encephalitis: inflammation of the brain; (4) headache: migraine, expansion of blood vessels pressing on nerves or constriction blocking blood supply, inflammation, muscle contraction to face, neck or scalp; (5) meningitis: infection of spinal fluid and meninges; (6) neuralgia: nerve pain, peripheral neuropathy, sciatica, shingles, trigeminal neuralgia; (7) Parkinson's disease: tremors in limbs, muscular rigidity; and (8) amnesia: loss of memory and inability to form new memory, and others such as ataxia, Bell's palsy, epilepsy, multiple sclerosis, myasthenia gravis, narcolepsy, paralysis, and rabies.

N-propanoyl derivatives of amino acids or aminocarbohydrates, and derivatives thereof, have broad utilities for prevention or treatment to alleviate or improve symptoms or syndromes associated with the vascular system. Vascular conditions, reactions and disorders include, but are not limited to, acanthosis nigricans, acrocyanosis, actinic cheilitis, actinic prurigo, dermatitis, dermatosis, dermographism, dyshidrosis, drug eruptions, eczema, erythema, erythema migrans, erythrocyanosis, erythromelalgia, familial hemorrhage, histamine reaction, inflammatory papular and pustular lesions, lichen planus, lupus erythematosus, mycosis fungoides, neurodermatitis, neuropeptide and neurovascular reactions, parapsoriasis, perniosis(chilblains), photoallergy, photoreaction, photosensitivity, pityriasis rosea, pityriasis rubra pilaris, polymorphic light eruption, psoriasis, rhinophyma, rosacea, sclerosis, spider naevi, T-cell disorders, telangiectasia, and urticaria and other vascular reactions.

N-propanoyl derivatives of amino acids or aminocarbohydrates, and derivatives thereof, have broad utilities for prevention or treatment to alleviate or improve symptoms or syndromes associated with the musculoskeletal system. Abnormalities of musculoskeletal system include, but are not limited to, (1) osteoporosis: reduction of calcium in bone leading to thin and susceptible to fracture, (2) osteoarthritis: inflammation of joint cartilage provoking swelling and pain, (3) rheumatoid arthritis: inflammation of synovium and destructions of cartilage, damage to heart, lungs, nerves and eyes, (4) ankylosing spondylitis: arthritis affecting sacroiliac joints and spine with inflammation and immovability, (5) bursitis: inflammation of bursa, (6) tendinitis: inflammation of tendon, (7) gout: recurrent acute arthritis from uric acid deposit, and others such as backache, bunion and hernia.

N-propanoyl derivatives of amino acids or aminocarbohydrates, and derivatives thereof, have broad utilities for prevention or treatment to alleviate or improve symptoms or syndromes associated with the cutaneous system. Disorders or abnormalities of the cutaneous system include, but are not limited to, disturbed keratinization, pigmentation and immunity; inflammation; infections and decreased physiological functions. The manifestations of cutaneous disorders may include acne; age spots; blemished skin; blotches; cellulite; dermatoses; dandruff; dry skin; pruritus, eczema; ichthyosis; keratoses and hyperkeratoses; lentigines; melasmas; mottled skin; pseudofolliculitis barbae; photoaging and photodamage; psoriasis; skin lines; stretch marks; thinning of skin, nail plate and hair; wrinkles; xerosis; oral or gum disease; irritated, inflamed, unhealthy, damaged or abnormal mucosa, skin, hair, nail, nostril, ear canal, anal or vaginal conditions; defective synthesis or repair of dermal components; abnormal or diminished synthesis of collagen, glycosaminoglycans, proteoglycans and elastin as well as diminished levels of such components in the dermis; uneven and rough surface of skin, nail and hair; loss or reduction of skin, nail and hair resiliency, elasticity and recoilability; lack of skin, nail and hair lubricants and luster; fragility and splitting of nail and hair; yellowing skin; reactive, irritating or telangiectatic skin; dull and older-looking skin, nail and hair; for skin bleach and lightening and wound healing.

In preferred embodiments, N-propanoyl derivatives of amino acids or aminocarbohydrates, and derivatives thereof, may be used to improve cognition and memory performance in Alzheimer's subjects, inflammatory and painful joints of osteoarthritis and rheumatoid arthritis, and deranged or disordered cutaneous tissues relevant to skin, nail and hair; oral, vaginal and anal mucosa; skin wound; disturbed keratinization; inflammation, and changes associated with intrinsic and extrinsic aging. The manifestations of cutaneous disorders include acne; age spots; blemished skin; blotches; cellulite; dermatoses; dandruff; dry skin; pruritus, eczema; ichthyosis; keratoses and hyperkeratoses; lentigines; melasmas; mottled skin; pseudofolliculitis barbae; photoaging and photodamage; psoriasis; skin lines; stretch marks; thinning of skin, nail plate and hair; wrinkles; xerosis; oral or gum disease; irritated, inflamed, unhealthy, damaged or abnormal mucosa, skin, hair, nail, nostril, ear canal, anal or vaginal conditions; defective synthesis or repair of dermal components; abnormal or diminished synthesis of collagen, glycosaminoglycans, proteoglycans and elastin as well as diminished levels of such components in the dermis; uneven and rough surface of skin, nail and hair; loss or reduction of skin, nail and hair resiliency, elasticity and recoilability; lack of skin, nail and hair lubricants and luster; fragility and splitting of nail and hair; yellowing skin; reactive, irritating or telangiectatic skin; dull and older-looking skin, nail and hair; for skin bleach and lightening and wound healing.

In accordance with preferred embodiments, N-propanoyl derivatives of amino acids or aminocarbohydrates, and derivatives thereof may be used to in the treatment of pains, pruritus, inflammation, erythema, dermatitis, acne, eczema, severe dry skin, ichthyosis, age spots, psoriasis, wrinkles, and photoaging skin.

In a preferred embodiment, the N-Propanoyl derivatives of amino acids and aminocarbohydrates, and preferably the N-propanoylamino acids, of the present invention, may be used for treating Alzheimer's disease. Specifically, N-propanoylamino acids such as N-propanoyl-L-glutamic acid and N-propanoyl-L-glutamine may be administered to a subject for the treatment of Alzheimer's disease. Such treatment may cause distinct improvement in multiple signs that gauge the severity status of Alzheimer's Disease.

A person skilled in the art will be capable of determining the optimal dosage, using the guidelines provided herein. The optimal dose is dependent, for example, by the progression of the disease and physical characteristics of the patient in need. The procedure for treating Alzheimer's disease may include administering a N-Propanoyl derivative of the present invention to a subject or patient in need thereof. The daily dosage of administered to a patient may be between about 10 mg to about 1000 mg, and preferably between about 100 mg to about 500 mg. For example, an N-propanoylamino acid, such as N-propanoyl-L-glutamic acid or N-propanoyl-L-glutamine, may be administered to a patient at a dose between between about 50 mg and about 100 mg, or twice daily at a dose between 25 mg and about 50 mg.

Furthermore, the dose administered to a subject may initially be a low dose, and then gradually increased to an optimally effective dose. For example, N-Propanoyl-L-glutamic acid 5 grams may be dissolved in ethanol with final volume of 100 ml to provide a 5% solution, 50 mg per ml. Initially, N-propanoyl-L-glutamic acid 25 mg in 0.5 ml ethanol may be added to fruit juice for convenience of oral administration. This dose of 25 mg preferably may be administered twice daily for 4 weeks, at which time the dose may be increased to 50 mg twice daily for 4 weeks. The patient undergoing treatment of Alzheimer's disease will show signs of regaining short term memory for events of the day within a several month treatment period.

In accordance with the preferred embodiments, vitamins and/or other pharmaceutical agents used to treat Alzheimer's disease may be used in combination with N-propanoyl derivatives of amino acids and aminocarbohydrates for the treatment of Alzheimer's disease. Examples of vitamins and pharmaceutical agent include, but are not limited to, donepezil, memantine, melatonin, lipoic acid, selenium, and folic acid. These agents may be used or taken simultaneously or sequentially with topical or oral administration of N-propanoyl derivatives of amino acids and aminocarbohydrates to provide complementary or enhancing effects.

For subjective disorders such as pain, itch, or the like, the therapeutic effects were evaluated or judged by the subjects or patients. For example, the subjects evaluated whether the pain or itch had disappeared within hours or days. For other detectable symptoms or syndromes, the therapeutic effects or improvements were evaluated or judged by medical professionals.

The following examples are illustrative, but not limiting, of the methods and compositions of the present invention. Other suitable modifications and adaptations of the variety of conditions and parameters normally encountered in therapy and that are obvious to those skilled in the art are within the spirit and scope of the embodiments.

### EXAMPLE 1

Skin thickness was measured by micrometer calipers to study skin changes associated with aging as follows: The skin was grasped with a 2×6 cm metal hinge, the internal faces of that were coated with emery cloth to prevent slippage, and manually squeezed to threshold subject discomfort. Combined thickness of two whole-skin layers including thickness of the two hinge leaves was measured with micrometer calipers. Thickness of the two hinge leaves was subtracted to determine the actual thickness of two whole-skin layers. Triplicate measurements on treated sites were done and an average number was used for calculation of the skin thickness.

### EXAMPLE 2

A typical N-propanoylamino acid in a cream composition was formulated as follows: N-Propanoyl-L-proline 5 g was dissolved in warm water 15 ml and propylene glycol 5 ml, and the solution thus obtained was mixed uniformly with hydrophilic ointment or oil-in-water emulsion 75 g. The cream thus formulated had pH 2.6 and contained 5% N-propanoyl-L-proline.

### EXAMPLE 3

N-Propanoyl-L-prolinamide 7 g was dissolved in warm water 20 ml and propylene glycol 10 ml, and the solution thus obtained was mixed uniformly with hydrophilic ointment or oil-in-water emulsion 63 g. The cream thus formulated had pH 4.5 and contained 7% N-propanoyl-L-prolinamide.

### EXAMPLE 4

N-Propanoyl-L-tyrosine 5 g was dissolved in warm ethanol 10 ml and propylene glycol 20 ml, and the solution thus obtained was mixed uniformly with hydrophilic ointment or oil-in-water emulsion 65 g. The cream thus formulated had pH 1,5 and contained 5% N-propanoyl-L-tyrosine.

### EXAMPLE 5

N-Propanoyl-L-methionine 5 g was dissolved in warm water 20 ml and propylene glycol 10 ml, and the solution thus obtained was mixed uniformly with hydrophilic ointment or oil-in-water emulsion 65 g. The cream thus formulated had pH 2.2 and contained 5% N-propanoyl-L-methionine.

### EXAMPLE 6

N-Propanoyl-L-arginine 5 g was dissolved in warm water 20 ml and propylene glycol 10 ml, and the solution thus obtained was mixed uniformly with hydrophilic ointment ointment or oil-in-water emulsion 65 g. The cream thus formulated had pH 4.3 and contained 5% N-propanoyl-L-arginine.

### EXAMPLE 7

N-Propanoyl-D-glucosamine 5 g was dissolved in water 15 ml and propylene glycol 5 ml, and the solution thus obtained was mixed uniformly with hydrophilic ointment or oil-in-water emulsion 75 g. The cream thus formulated had pH 4.7 and contained 5% N-propanoyl-D-glucosamine.

A male subject, age 69, having an itchy lesion on his right foot due to atopic eczema, topically applied the above cream to the lesion. A few minutes after the topical application, the itch disappeared completely and the skin remained free of itch for the following 12 hours.

### EXAMPLE 8

N-Propanoyl-L-glutamic acid 5 g was dissolved in warm water 20 ml and propylene glycol 15 ml. Arginine 2 g was added to make an amphoteric system, and the solution thus obtained was mixed uniformly with hydrophilic ointment or oil-in-water emulsion 58 g. The cream thus formulated had pH 5.1 and contained 5% N-propanoyl-L-glutamic acid in an amphoteric composition.

### EXAMPLE 9

N-Propanoyl-creatinine 5 g was dissolved in warm water 15 ml and propylene glycol 10 ml, and the solution thus obtained was mixed uniformly with hydrophilic ointment or oil-in-water emulsion 70 g. The cream thus formulated had pH 4.8 and contained 5% N-propanoyl-creatinine.

### EXAMPLE 10

A female subject, age 54, applied topically twice daily 5% N-propanoyl-D-glucosamine cream as formulated in Example 7 to fine wrinkles (crow's feet) near the eyes for five weeks. After five weeks of topical treatment, her wrinkles improved significantly.

### EXAMPLE 11

N-Propanoyl-L-prolinamide 5 g was dissolved in 95 ml solution prepared from water 40 parts, ethanol 40 parts and propylene glycol 20 parts by volume. The solution thus prepared had pH 4.9 and contained 5% N-propanoyl-L-prolinamide.

A female subject, age 20, who had adolescent acne with multiple papules and pustules on her face, applied topically twice daily the above composition containing 5% N-propanoyl-L-prolinamide. After two weeks of treatment, most lesions became less inflamed and improved substantially.

### EXAMPLE 12

N-Propanoyl-D-glucosamine 10 g was dissolved in 90 ml solution prepared from water 40 parts, ethanol 40 parts and propylene glycol 20 parts by volume. The formulation thus prepared had pH 6.0 and contained 10% N-propanoyl-D-glucosamine solution.

### EXAMPLE 13

A female subject, age 63, applied topically twice daily N-propanoyl-D-glucosamine 10% solution of Example 12 to her left forearm for a total of 9 weeks. After 4 weeks there was no change in skin thickness of her untreated right forearm, her left forearm had increased 8% in skin thickness as measured by the micrometer calipers of Example 1. After 6 weeks her left forearm had increased 23% and after 9 weeks 28% in skin thickness while there was no change in skin thickness of her right forearm. At the end of 9 weeks her untreated right forearm was still loose, relatively thin and wrinkled when lifted. In contrast, her left forearm was firmer, smooth, plump and minimally wrinkled when lifted. This result indicated that N-propanoyl-D-glucosamine would be therapeutically effective for topical treatment of wrinkles and changes of skin, nail or hair associated with aging.

### EXAMPLE 14

A female subject, age 45, applied topically twice daily N-propanoyl-D-glucosamine 10% solution of Example 12 to her left forearm for a total of 8 weeks. After 5 weeks there was no change in skin thickness of her untreated right forearm, her left forearm had increased 19% in skin thickness as measured by the micrometer calipers of Example 1. After 8 weeks her left forearm had increased 33% in skin thickness. At the end of 8 weeks her untreated right forearm was still loose, relatively thin and wrinkled when lifted. In contrast, her left forearm was firmer, smooth, plump and minimally wrinkled when lifted. This result indicated that N-propanoyl-D-glucosamine would be therapeutically effective for topical treatment of wrinkles and changes of skin, nail or hair associated with aging.

### EXAMPLE 15

A female subject, age 85, applied topically twice daily N-propanoyl-D-glucosamine 10% solution of Example 12 to her right forearm for a total of 6 weeks. After 4 weeks there was no change in skin thickness of her untreated left forearm, her right forearm had increased 13% in skin thickness as measured by the micrometer calipers of Example 1. After 6 weeks her right forearm had increased 27% in skin thickness. At the end of 6 weeks her untreated left forearm was still loose, relatively thin and wrinkled when lifted. In contrast, her right forearm was firmer, smooth, plump and minimally wrinkled when lifted. This result indicated that N-propanoyl-D-glucosamine would be therapeutically effective for topical treatment of wrinkles and changes of skin, nail or hair associated with aging.

### EXAMPLE 16

A human subject having itchy skin from atopic eczema topically applied 5% N-propanoyl-glucosamine cream to the lesion. A few minutes after the topical application, the itch disappeared completely and the skin remained free of itch for the following 12 hours.

### EXAMPLE 17

A human subject with photoaging skin topically applied twice daily 10% N-propanoyl-glucosamine solution to her left forearm for 8 weeks. At the end of 8 weeks her untreated right forearm was still loose, relatively thin and wrinkled when lifted. In contrast, her treated left forearm was firmer, smooth, plump, minimally wrinkled when lifted, and had increased 33% in skin thickness. This result indicated that N-propanoyl-glucosamine would be therapeutically effective for topical treatment of skin, nail, or hair for wrinkles and changes associated with aging.

While the general embodiments have been described with reference to particularly preferred embodiments and examples, those skilled in the art recognize that various modifications may be made without departing from the spirit and scope thereof.

What is claimed is:

1. A composition suitable for systemic or topical administration to a subject comprising:
a therapeutically effective amount of an N-propanoyl derivative selected from the group consisting of N-propanoylamino acids, N-propanoylaminocarbohydrates, and derivatives thereof, including all stereoisomers and mixtures thereof;
wherein the N-propanoyl derivative is in the form of a free acid, salt, partial salt with organic or inorganic alkali, amide, ester, or lactone; and
wherein the N-propanoyl derivative is not N-propanoyl-proline or N-propanoyl-hydroxyproline.

2. The composition of claim 1, wherein the N-propanoyl derivative is selected from the group consisted of N-propanoyl-alanine, N-propanoyl-arginine; N,N′-dipropanoyl-arginine, N-propanoyl-asparagine, N-propanoyl-aspartic acid, N-propanoyl-cysteine, N-propanoyl-glycine, N-propanoyl-glutamic acid, N-propanoyl-glutamine, N-propanoyl- histidine; N,N -dipropanoyl-histidine, N-propanoyl-isoleucine, N-propanoyl-leucine, N-propanoyl-lysine; N,N-dipropanoyl-lysine, N-propanoyl-methionine, N-propanoyl-phenylalanine, N-propanoyl-serine, N-propanoyl-threonine, N-propanoyl-tryptophan; N,N-dipropanoyl-tryptophan, N-propanoyl-tyrosine, N-propanoyl-valine, N-propanoyl-β-alanine, N-propanoyl-γ-aminobutanoic acid, N-propanoyl-β-aminoisobutanoic acid, N-propanoyl-citrulline, N-propanoyl-dopa (N-propanoyl-3,4-dihydroxyphenylalanine), N-propanoyl-homocysteine, N-propanoyl-homoserine, N-propanoyl-ornithine, and N,N′-dipropanoyl-ornithine.

3. The composition of claim 1, wherein the N-propanoyl derivative is selected from the group consisted of N-propanoyl-glycerosamine, N-propanoyl-erythrosamine, N-propanoyl-threosamine, N-propanoyl-ribosamine, N-propanoyl-arabinosamine, N-propanoyl-xylosamine, N-propanoyl-lyxosamine, N-propanoyl-allosamine, N-propanoyl-altrosamine, N-propanoyl-glucosamine, N-propanoyl-mannosamine, N-propanoyl-gulosamine, N-propanoyl-idosamine, N-propanoyl-galactosamine, N-propanoyl-talosamine, N-propanoyl-alloheptosamine, N-propanoyl-altroheptosamine, N-propanoyl-glucoheptosamine, N-propanoyl-mannoheptosamine, N-propanoyl-guloheptosamine, N-propanoyl-idoheptosamine, N-propanoyl-galactoheptosamine, N-propanoyl-taloheptosamine, N-propanoyl-glyceraminic acid, N-propanoyl-erythrosaminic acid, N-propanoyl-threosaminic acid, N-propanoyl-ribosaminic acid, N-propanoyl-arabinosaminic acid, N-propanoyl-xylosaminic acid and N-propanoyl-lyxosaminic acid.

4. The composition of claim 1, wherein the N-propanoyl derivative is selected from the group consisted of N-propanoyl-allosaminic acid, N-propanoyl-altrosaminic acid, N-propanoyl-glucosaminic acid, N-propanoyl-mannosaminic acid, N-propanoyl-gulosaminic acid, N-propanoyl-idosaminic acid, N-propanoyl-galactosaminic acid, N-propanoyl-talosaminic acid, N-propanoyl-alloheptosaminic acid, N-propanoyl-altroheptosaminic acid, N-propanoyl-glucoheptosaminic acid, N-propanoyl-mannoheptosaminic acid, N-propanoyl-guloheptosaminic acid, N-propanoyl-idoheptosaminic acid, N-propanoyl-galactoheptosaminic acid, N-propanoyl-taloheptosaminic acid, N-propanoyllactosamine, N-propanoylmuramic acid, N-propanoylneuramine, N-propanoyineuramin lactose, N-propanoyl-neuraminic acid. N-propanoyl-glycerosylamine, N-propanoyl-erythrosylamine, N-propanoyl-threosylamine, N-propanoyl-ribosylamine, N-propanoyl-arabinosylamine, N-propanoyl-xylosylamine, N-propanoyl-lyxosylamine, N-propanoyl-allosylamine, N-propanoyl-altrosylamine, N-propanoyl-glucosylamine, N-propanoyl-mannosylamine, N-propanoyl-gulosylamine, N-propanoyl-idosylamine, N-propanoyl-galactosylamine, N-propanoyl-talosylamine, N-propanoyl-alloheptosylamine, N-propanoyl-altroheptosylamine, N-propanoyl-glucoheptosylamine, N-propanoyl-mannoheptosylamine, N-propanoyl-guloheptosylamine, N-propanoyl-idoheptosylamine, N-propanoyl-galactoheptosylamine, N-propanoyl-taloheptosylamine, and N-propanoyl-aminocyclitols.

5. The composition of claim 1, wherein the N-propanoyl derivative is selected from the group consisted of N-propanoyl-prolinamide, N-propanoyl-proline ethyl ester, N-propanoyl-proline propyl ester, N-propanoyl-glutamic acid, N-propanoyl-glutamic acid diethyl ester, N-propanoyl-glutamine, N-propanoyl-glutamine ethyl ester, N-propanoyl-glucosamine, N-propanoyl-galactosamine and N-propanoyl-creatinine.

6. The composition of claim 1, wherein the N-propanoyl derivative is selected from the group consisting of N-propanoyl-prolinamide, N-propanoyl-proline ester, and derivatives thereof.

7. The composition of claim 1, wherein the N-propanoyl derivative is a N-propanoylamino acid, including all stereoisomers and mixtures thereof, comprising the structure represented by:
R₁CH(NHR₂)(CH₂)ₙCOR₃,
where R₁ is H, an alkyl, aralkyl or aryl group having 1 to 14 carbon atoms, and R₁ may also carry OH, SH, SCH₃, NH₂, NHR₂, CONH₂, NHCONH₂, NHC(═NH)NH₂, NHC(═NR₂)NH₂, imidazole, pyrrolidine or other heterocyclic group; n is an integer from 0 to 5; R₂ is independently an propanoyl group having COCH₂CH₃; R₃ is NH₂, OR₄; R₄ is H, an alkyl, aralkyl or aryl group having 1 to 9 carbon atoms; and where the H attached to any carbon atom is optionally substituted with an I, F, Cl, Br, OH, or alkoxy group having 1 to 9 carbons.

8. The composition of claim 7, wherein the N-propanoylamino acid is in the form of a free acid, salt, partial salt with organic or inorganic alkali amide, ester, or lactone.

9. The composition of claim 7, wherein the N-propanoylamino acid is a N-propanoyl derivative selected from the group consisting of N-propanoyl-glutamic acid, N-propanoyl-glutamic acid ester, N-propanoyl-glutamic diester, and derivatives thereof.

10. The composition of claim 1, wherein the N-propanoyl derivative is a N-propanoylaminocarbohydrate, including all stereoisomers and mixtures thereof, comprising a structure represented by:
R₁(CHOH)ₙ(CHNHR₂)R₃,
where R₁ is selected from the group consisting of H, I, F, Cl, Br, CHO, CONH₂, COOR₄, an alkyl, alkoxyl, aralkyl or aryl group having 1 to 9 carbon atoms; n is an integer, preferably from 1-9; R₂ is a propanoyl group having COCH₂CH₃; R₃ is selected from the group consisting of H, CHO, CONH₂, and COOR₄; R₄ is independently selected from the group consisting of H, an alkyl, aralkyl or aryl group having 1 to 9 carbon atoms; and where the H attached to any carbon atom is optionally substituted with an I, F, Cl, Br, SH, CHO, CONH₂, NH₂, or an alkyl, alkoxyl, aralkyl, or aryl group having 1 to 9 carbon atoms.

11. The composition of claim 10, wherein the N-propanoylaminocarbohydrate is in the form of a free acid, salt, partial salt with organic or inorganic alkali amide, ester, or lactone.

12. The composition of claim 10, wherein the aminocarbohydrate of the N-propanoylaminocarbohydrate comprises only one amino group.

13. The composition of claim 1, further comprising an additional agent selected from the group consisting of vitamins, cosmetics, and pharmaceutical agents.

14. The composition of claim 13, wherein the additional agent is selected from the groups consisting of hydroxyacids, ketoacids and related compounds; phenyl alpha acyloxyalkanoic acids and derivatives; N-acetyl-aldosamines, N-acetylamino acids and related N-acetyl compounds; local analgesics and anesthetics; antiacne agents; antibacterials; antiyeast agents; antifungal agents; antiviral agents; antidandruff agents; antidermatitis agents; antihistamine agents; antipruritic agents; antiemetics; antimotion sickness agents; antiinflammatory agents; antihyperkeratotic agents; antiperspirants; antipsoriatic agents; antiseborrheic agents; hair conditioners and hair treatment agents; antiaging and antiwrinkle agents; sunblock and sunscreen agents; skin lightening agents; depigmenting agents; astringents; cleansing agents; corn, callus and wart removing agents; topical cardiovascular agents; vitamins; corticosteroids; tanning agents; hormones; retinoids; and gum disease or oral care agents.

15. The composition of claim 1, formulated into a form suitable for topical administration selected from the group consisting of a solution, gel, lotion, cream, ointment, shampoo, spray, stick, powder, masque, mouth rinse or wash, and vaginal gel or preparation.

16. The composition of claim 1, formulated into a form suitable for systemic administration to a subject, wherein the route of systemic administration is by injection, infusion, or oral administration.

17. A method of preventing or treating a disorder or syndrome comprising systemically or topically administering the composition of claim 1 to a subject, wherein the disorder or syndrome is associated with any one of the nervous, vascular, musculoskeletal, or cutaneous systems.

18. The method of claim 17, wherein the symptom or syndrome associated with the nervous system is Alzheimer's disease.

19. The method of claim 17, wherein the symptom or syndrome is selected from the group consisting of pains, pruritus, inflammation, erythema, dermatitis, acne, eczema, severe dry skin, ichthyosis, age spots, psoriasis, wrinkles, and photoaging skin.

20. The method of claim 17, wherein the symptom or syndrome associated with musculoskeletal is selected from the group consisted of osteoporosis, osteoarthritis, rheumatoid arthritis, ankylosing spondylitis, bursitis, and tendonitis.

21. The method of claim 17, wherein the composition is administered orally.

22. A method of preventing or treating a disorder or syndrome comprising systemically or topically administering the composition of claim 6 to a subject, wherein the disorder or syndrome is associated with any one of the nervous, vascular, musculoskeletal, or cutaneous systems.
